(19) United States
(12) Patent Application Publication
(10) Pub. No.: US 2023/0263789 A1
(43) Pub. Date: Aug. 24, 2023

(54) PHARMACEUTICAL COMPOSITION AND PREPARATION METHOD

(71) Applicant: JIANGSU KANION PHARMACEUTICAL CO., LTD., Lianyungang, Jiangsu (CN)

(72) Inventors: Wei XIAO, Lianyungang, Jiangsu (CN); Hui ZHANG, Lianyungang, Jiangsu (CN); Qingming GUO, Lianyungang, Jiangsu (CN); Zhenzhong WANG, Lianyungang, Jiangsu (CN); Shasha GU, Lianyungang, Jiangsu (CN); Xiaolian HE, Lianyungang, Jiangsu (CN); Xuehong DONG, Lianyungang, Jiangsu (CN); Yanqiu WANG, Lianyungang, Jiangsu (CN)

(73) Assignee: JIANGSU KANION PHARMACEUTICAL CO., LTD., Lianyungang, Jiangsu (CN)

(21) Appl. No.: 18/005,237

(22) PCT Filed: May 19, 2021
(86) PCT No.: PCT/CN2021/094653

§ 371 Date: Jan. 12, 2023

(30) Foreign Application Priority Data
Jul. 17, 2020 (CN) 202010693600.6

(51) Int. Cl. A61K 31/444; A61K 9/16

(52) U.S. Cl. CPC A61K 31/444; A61K 9/1635

(57) ABSTRACT

A pharmaceutical composition that includes: an active ingredient, including 2-(2-aminopyridine-4-yl)-5-(5-((5-(2-ethyl-2H-tetrazole-5-yl)pyridine-2-yl)-oxy)-3,3-amyldimethyl)-1,2,5-thiadiazolidin 1,1-dioxide or pharmaceutically acceptable salt thereof; and auxiliary materials, including a diluent and a suspending agent, where the suspending agent includes povidone. Furthermore, the pharmaceutical composition is rapid in dissolution, high in stability, simple in preparation process and more suitable for industrialized mass production.

### TECHNICAL FIELD

The present invention belongs to the field of pharmaceutical preparations, and particularly relates to a pharmaceutical composition for treating child hand-foot-and-mouth disease, and a preparation method thereof.

### BACKGROUND

Hand-foot-and-mouth disease (HFMD) is a global infectious disease caused by enterovirus. The main pathogenic viruses include enterovirus 71 (EV71), Coxsachie A16 (CoxA16) and other enteroviruses, where EV71 is the most harmful. The hand-foot-and-mouth disease is more common in infants and children, especially children under 5 years old, but less common in adults. However, patients with immunodeficiency may also be infected. At onset, blisters on hands, feet and mouth are the typical features. In addition, the disease is accompanied by symptoms such as fever, headache, sore throat and rash, etc. In severe cases, myocarditis, pulmonary edema, aseptic meningitis and even fatal pulmonary embolism or massive hemorrhage may occur. Individual severe children's conditions develop rapidly and the mortality rate is high.

The compound has been disclosed at least in two prior art documents [CN105085512A and CN106661009A]. Both of the documents have disclosed the structure and use of the compound to treat hand foot mouth disease caused by enterovirus 71 and the preparation method thereof.

Specifically, the last chemical structure in the ninth page of the CN106661009A is the structure of the compound and its Embodiment 75F (100 pages) discloses the preparation method and nuclear magnetic hydrogen spectrum of the compound. The last chemical structure of para [0055] discloses the structure of the compound, and the preparation method and nuclear magnetic hydrogen spectrum of the compound are disclosed in [1168]-[1171] paras of CN105085512A.

### SUMMARY

An objective of the present invention is to provide a pharmaceutical composition with high stability and rapid dissolution and for treating child hand-foot-and-mouth disease, and a preparation method thereof. Specifically, the pharmaceutical composition provided by the present invention includes:

an active ingredient, including 2-(2-aminopyridine-4-yl)-5-(5-((5-(2-ethyl-2H-tetrazole-5-yl)pyridine-2-yl)-oxy)-3,3-amyldimethyl)-1,2,5-thiadiazolidin 1,1-dioxide or pharmaceutically acceptable salt thereof; and

auxiliary materials, including a diluent and a suspending agent, where the suspending agent includes povidone.

Further, the suspending agent further includes one or more of carboxymethylcellulose sodium, carrageenan or crospovidone (CL-M).

Specifically, the pharmaceutically acceptable salt includes hydrochloride, sulfate, citrate, benzenesulfonate, hydrobromide, hydrofluoric acid, phosphate, acetate, propionate, succinate, oxalate, malate, fumarate, maleate, tartrate or trifluoroacetate.

Optionally, the povidone is povidone K30 or povidone K90.

Further, the content of the suspending agent povidone K90 is 0.3% to 2.0% of a total weight of the composition, preferably, 0.5 to 1.5%.

Further, the content of the suspending agent crospovidone (CL-M) is 0.5% to 2.0% of a total weight of the composition, preferably, 0.8 to 1.5%.

Further, the pharmaceutical composition further includes a solubilizer. The solubilizer is preferably poloxamer 188 and/or polysorbate 80 and/or tween 80; and the solubilizer preferably accounts for 0.5% to 2.0% of a total weight of the pharmaceutical composition, preferably, 0.8 to 1.5%.

Further, the diluent includes one or more of sucrose, mannitol, sorbitol and xylitol.

Preferably, the diluent includes mannitol and sorbitol; and the weight ratio of the mannitol to the sorbitol is 1:1-3:1.

Further, the auxiliary materials further include essence; the essence includes one or more of orange essence, strawberry essence and banana essence; and the essence preferably accounts for 0% to 1.0% of a total weight of the pharmaceutical composition, preferably, 0.1% to 0.5%.

Further, the active ingredient accounts for 1.0% to 3.0% of a total weight of the pharmaceutical composition; the diluent accounts for 60% to 98% of the total weight of the pharmaceutical composition; and the suspending agent accounts for 0.3% to 2.0% of the total weight of the pharmaceutical composition. When the pharmaceutical composition is a granule, the specific weight of each bag of active ingredient is 0.1 mg to 200 mg, preferably, 1 mg to 100 mg, more preferably, 10 mg to 100 mg, most preferably, 50 mg.

Further, the diluent accounts for 80% to 95% of a total weight of the pharmaceutical composition.

Preferably, the pharmaceutical composition provided by the present invention includes: 1 part of compound A, 26 parts of mannitol, 12 parts of sorbitol and 0.2 parts to 0.3 parts of povidone K30 or povidone K90.

Further, the pharmaceutical composition further includes: 0.4 parts of poloxamer 188.

Further, the pharmaceutical composition further includes: 0.4 parts of crospovidone (CL-M).

Further, the pharmaceutical composition further includes: 0.08 parts of strawberry essence.

Specifically, the pharmaceutical composition is a granule prepared from the active ingredient and the auxiliary materials through mixing, granulation, drying, granule arrangement, total mixing and packaging.

The present invention further provides a use of the pharmaceutical composition for treating child hand-foot-and-mouth disease.

The present invention further provides a method for preparing the pharmaceutical composition as described in any one of the above. The method includes:

a) obtaining an adhesive;

b) mixing an active ingredient and a filling agent, and adding the adhesive for granulation;

c) performing dynamic drying; and

d) performing granule arrangement and total mixing.

Further, when the pharmaceutical composition is selected from a granule, the method includes:

a) adding 0.4 parts of poloxamer 188 and 0.3 parts of povidone K90 into a 80% ethanol solution for uniform mixing to serve as the adhesive;

b) weighing 2-(2-aminopyridine-4-yl)-5-(5-((5-(2-ethyl-2H-tetrazole-5-yl)pyridine-2-yl)-oxy)-3,3-amyldimethyl)-1,2,5-thiadiazolidin 1,1-dioxide hydrochloride, mannitol, sorbitol and crospovidone (CL-M) according to a weight ratio of 1:26:12:0.4 by part, mixing the materials uniformly and then adding the adhesive to obtain a soft material, and performing sieving with a 20-mesh sieve for granulation;

c) performing drying at 50° C.; and

d) performing granule arrangement with a 20-mesh sieve, adding essence into the granules after granule arrangement for total mixing, and packaging.

According to the present invention, the pharmaceutical composition with rapid dissolution and high stability is finally obtained by screening the types and the dosage ratio of the auxiliary materials; and the pharmaceutical composition is simple in preparation process and more suitable for industrialized mass production.

### DETAILED DESCRIPTION OF THE EMBODIMENTS

An active substance (compound A: 2-(2-aminopyridine-4-yl)-5-(5-((5-(2-ethyl-2H-tetrazole-5-yl)pyridine-2-yl)-oxy)-3,3-amyldimethyl)-1,2,5-thiadiazolidin 1,1-dioxide hydrochloride) of the present invention can lock viruses by entering a pocket cavity of an EV71 virus nucleocapsid to prevent a viral capsid from decomposing and releasing virus RNA, thereby inhibiting further replication of the virus. The structural formula is as follows:

Chemical Structural Formula:

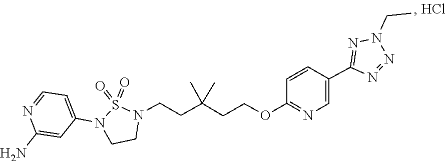 [SMILES: CCn1nnc(-c2ccc(OCCC(C)(C)CCN3CCN(c4ccnc(N)c4)S3(=O)=O)nc2)n1]

molecular formula: C₂₂H₃₁N₉O₃S·HCl

molecular weight: 538.07

To improve the solubility of insoluble medicines, surfactants, acid-base regulators and the like are mostly used in the prior art. According to the present invention, it is found in the preliminary test process that a compound A and an acidic regulator (citric acid or tartaric acid) can realize complete dissolution of the compound A; upon further study, it is found that when the adding amount of the acid is 0.2 to 1.0 that of the compound A, the solubility of the compound A in hot water can be improved. However, the granules obtained in the preliminary study process can solve the problem of solubility, but the stability is insufficient. Therefore, how to improve the solubility of the active substance provided by the present invention and ensure the stability has become a difficult point in a medicine technology.

In view of this, the present invention provides other preparation forms of the pharmaceutical composition for treating child hand-foot-and-mouth disease, thereby solving the problem of the solubility and the stability of the compound by selecting appropriate auxiliary materials.

The present invention is further described below by the following embodiments and test examples. The embodiments and test examples are only used to illustrate the objective, but not to limit the scope of the present invention.

In order to enable those skilled in the art to more clearly understand the technical solution of the present application, the technical solution of the present application will be described in detail below with reference to specific embodiments.

The test materials used in the embodiments of the present invention are all conventional test materials in the field, which can be purchased through commercial channels or can be prepared by methods in the prior art.

Embodiment 1: 1 part of compound A, 26 parts of mannitol, 12 parts of sorbitol, 0.2 parts of carboxymethylcellulose sodium and 0.08 parts of strawberry essence;

Embodiment 2: 1 part of compound A, 26 parts of mannitol, 12 parts of sorbitol, 0.2 parts of hydroxypropyl methylcellulose and 0.08 parts of strawberry essence;

Embodiment 3: 1 part of compound A, 26 parts of mannitol, 12 parts of sorbitol, 0.2 parts of povidone K30 and 0.08 parts of strawberry essence;

Embodiment 4: 1 part of compound A, 26 parts of mannitol, 12 parts of sorbitol, 0.2 parts of povidone K90 and 0.08 parts of strawberry essence;

Embodiment 5: 1 part of compound A, 26 parts of mannitol, 12 parts of sorbitol, 0.2 parts of povidone K90, 0.4 parts of poloxamer 188 and 0.08 parts of strawberry essence;

Embodiment 6: 1 part of compound A, 26 parts of mannitol, 12 parts of sorbitol, 0.3 parts of povidone K90, 0.4 parts of poloxamer 188 and 0.08 parts of strawberry essence;

Embodiment 7: 1 part of compound A, 26 parts of mannitol, 12 parts of sorbitol, 0.3 parts of povidone K90, 0.4 parts of poloxamer 188, 0.4 parts of crospovidone (CL-M) and 0.08 parts of strawberry essence;

Embodiment 8: 1 part of compound A, 26 parts of mannitol, 12 parts of sorbitol, 0.2 parts of povidone K90, 0.1 part of carboxymethylcellulose sodium and 0.08 parts of strawberry essence; and

Embodiment 9: 1 part of compound A, 26 parts of mannitol, 12 parts of sorbitol, 0.2 parts of povidone K90, 0.3 parts of crospovidone (CL-M), 0.4 parts of polysorbate 80 and 0.08 parts of strawberry essence.

Preparation processes of Embodiments 1-9:

\textcircled{1} carboxymethylcellulose sodium/hydroxypropyl methylcellulose/povidone K30/povidone K90/poloxamer 188/polysorbate 80 were weighed according to a formula and were dissolved into 80% ethanol to prepare an adhesive.

\textcircled{2} A compound A and auxiliary materials mannitol/sorbitol/crospovidone (CL-M) were mixed uniformly, and the adhesive was added to prepare a soft material.

\textcircled{3} Granulation was performed with a 20-mesh sieve.

\textcircled{4} Drying was performed at 50° C. and the water content was controlled to be less than 2.0%.

\textcircled{5} Granule arrangement was performed with a 20-mesh sieve, and fine powder was removed with a 80-mesh sieve.

\textcircled{6} Essence was added and then total mixing was performed.

\textcircled{7} Granules after total mixing were collected.

\textcircled{8} Internal packaging was performed.

Comparative example 1: 1 part of compound A, 38 parts of sucrose, 0.2 parts of tartaric acid and 0.08 parts of strawberry essence.

A preparation process of Comparative Example 1:

\textcircled{1} tartaric acid was weighed and dissolved in 80% ethanol to prepare an adhesive.

\textcircled{2} A compound A and other auxiliary materials (except the tartaric acid and essence) were mixed uniformly, and the adhesive was added to prepare a soft material.

\textcircled{3} Granulation was performed with a 20-mesh sieve.

\textcircled{4} Drying was performed at 50° C. and the water content was controlled to be less than 2.0%.

\textcircled{5} Granule arrangement was performed with a 20-mesh sieve, and fine powder was removed with a 80-mesh sieve.

\textcircled{6} Essence was added and then total mixing was performed.

\textcircled{7} Granules after total mixing were collected.

\textcircled{8} Internal packaging was performed.

Comparative example 2: 1 part of compound A, 38 parts of sucrose, 0.6 parts of tartaric acid and 0.08 parts of strawberry essence.

A preparation process of Comparative Example 2 is the same as that of Comparative example 1.

Comparative example 3: 1 part of compound A, 38 parts of sucrose, 0.6 parts of tartaric acid, 0.4 parts of polysorbate 80 and 0.08 parts of strawberry essence.

A preparation process of Comparative Example 3 is the same as that of Comparative example 1.

1. Solubility Investigation

Granules prepared by the embodiments and the comparative examples were added into hot water to investigate the solubility. The results are shown in Table 1.

TABLE 1
The investigation results of the solubility

| | Granule | Dissolution | |
|---|---|---|---|
| Cases | Categories | Time | Dissolution Phenomenon |
| Embodiment 1 | Suspension | / | Flocculent precipitate |
| | granule | | |
| Embodiment 2 | Suspension | / | Flocculent precipitate |
| | granule | | |
| Embodiment 3 | Suspension | 18 s | The solution is a milky |
| | granule | | solution, stands for 3 min for |
| | | | settlement, and is shaken for |
| | | | 1 min, which is dispersed |
| | | | non-uniformly. (Heat |
| | | | preservation at 40° C. ) |
| Embodiment 4 | Suspension | 35 s | The solution is a milky |
| | granule | | solution, stands for 8 min for |
| | | | settlement, and is shaken for |
| | | | 1 min, which has low |
| | | | dispersibility. (Heat |
| | | | preservation at 40° C.) |
| Embodiment 5 | Suspension | 15 s | The solution is a |
| | granule | | semi-transparent and uniform |
| | | | milky solution, stands for 10 |
| | | | min for settlement, and is |
| | | | shaken for 1 min, which has |
| | | | low dispersibility. (Heat |
| | | | preservation at 40° C.) |
| Embodiment 6 | Suspension | 10 s | The solution is a |
| | granule | | semi-transparent and uniform |
| | | | milky solution, stands for 30 |
| | | | min for settlement, and is |
| | | | shaken for 1 min, which is |
| | | | dispersed uniformly. (Heat |
| | | | preservation at 40° C.) |
| Embodiment 7 | Suspension | 7 s | The solution is a |
| | granule | | semi-transparent and uniform |
| | | | milky solution, stands for 45 |
| | | | min for settlement, and is |
| | | | shaken for 20 s, which is |
| | | | dispersed uniformly. (Heat |
| | | | preservation at 40° C.) |
| Embodiment 8 | Suspension | 35 s | The solution is a milky |
| | granule | | solution and is shaken for 1 |
| | | | min, which has low |
| | | | dispersibility. (Heat |
| | | | preservation at 40° C.) |
| Embodiment 9 | Suspension | 12 s | The solution is a |
| | granule | | semi-transparent and uniform |
| | | | milky solution, stands for 45 |
| | | | min for settlement, and is |
| | | | shaken for 20 s, which is |
| | | | dispersed uniformly and is |
| | | | slightly stink. (Heat |
| | | | preservation at 40° C.) |
| Comparative | Soluble | 16 s | The solution is completely |
| example 1 | granule | | dissolved and is slightly |
| | | | turbid. |
| Comparative | Soluble | 8 s | The solution is completely |
| example 2 | granule | | dissolved, and is clear and |
| | | | transparent. |
| Comparative | Soluble | 6 s | The solution is completely |
| example 3 | granule | | dissolved, and is clear and |
| | | | transparent. |

According to the investigation results of the solubility, flocculent precipitates are found in Embodiments 1 and 2; suspension milky solutions are found in the embodiments or comparative examples in which povidone is added; semi-transparent milky solutions dispersed uniformly are found in Embodiment 6 and Embodiment 7; the solution in Embodiment 7 can be dispersed rapidly and uniformly through shaking after settlement; and polysorbate 80 is added in Embodiment 9 and the solution is slightly stink, and Embodiment 7 is finally preferred in view of the odor.

2. Stability Investigation

According to the guide principle of stability test of crude medicines and pharmaceutical preparations in Appendix XIXC of PHARMACOPOEIA OF THE PEOPLE'S REPUBLIC OF CHINA, 2015, Volume II, in the present invention, suspension granules in Embodiments 6 and 7, and soluble granules in Comparative Examples 2 and 3 were investigated by accelerated stability test and long-term stability test (Embodiment 7 and Comparative Example 3 were merely listed below).

The condition of the accelerated stability test: the pharmaceutical compositions prepared in Embodiment 7 and Comparative example 3 were respectively packaged according to a composite film for packaging a polyester/aluminum/polyethylene medicine product, and stood for 6 months under the temperature of 40° C. and the relative humidity of 75%, sampling was performed in the 0^{th}, 1^{st}, 3^{rd} and 6^{th} months during the test, and inspection was performed according to stability investigation items. The inspection results of the accelerated test of “related substances” are shown in Table 2. The results show that impurities in Comparative example 3 are higher than those in Embodiment 7, which indicates that the solubility of the formula in the comparative example meets the requirement, but the stability is insufficient. Similarly, the change of related substances of the suspension granules in Embodiment 6 is much smaller than those in Comparative examples 2 and 3.

TABLE 2
Inspection result of related substances of accelerated stability test

| | | Related Substances (%) | |
|---|---|---|---|
| | | Embodiment | Comparative |
| Inspection Items | | 7 | example 3 |
| 0^{th} month | Largest single | 0.05 | 0.07 |
| | Impurity | | |
| | Total impurities | 0.05 | 0.30 |
| 1^{st} month | Largest single | 0.09 | 0.25 |
| | Impurity | | |
| | Total impurities | 0.20 | 0.53 |
| 2^{nd} month | Largest single | 0.15 | 0.36 |
| | Impurity | | |
| | Total impurities | 0.31 | 0.59 |
| 3^{rd} month | Largest single | 0.13 | 0.52 |
| | Impurity | | |
| | Total impurities | 0.28 | 0.81 |
| 6^{th} month | Largest single | 0.16 | 0.77 |
| | Impurity | | |
| | Total impurities | 0.41 | 1.24 |

The condition of the long-term stability test: the pharmaceutical compositions prepared in Embodiment 7 and Comparative example 3 were respectively packaged according to a composite film for packaging a polyester/aluminum/polyethylene medicine product, and stood for 24 months under the temperature of 25° C. and the relative humidity of 60%, sampling was performed in the 0^{th}, 1^{st}, 3^{rd}, 6^{th}, 12^{th}, 18^{th} and 24^{th} months during the test, 12-month stability test was completed at present, and inspection was performed according to stability investigation items, where the inspection results of long-term test of “related substances” are shown in Table 3.

TABLE 3
Inspection result of related substances of long-term stability test

| | | Related Substances (%) | |
|---|---|---|---|
| | | | Comparative |
| Inspection Items | | Embodiment 7 | example 3 |
| 0^{th} month | Largest single | 0.05 | 0.07 |
| | Impurity | | |
| | Total impurities | 0.05 | 0.30 |
| 3^{rd} month | Largest single | 0.10 | 0.07 |
| | Impurity | | |
| | Total impurities | 0.15 | 0.31 |
| 6^{th} month | Largest single | 0.10 | 0.09 |
| | Impurity | | |
| | Total impurities | 0.23 | 0.36 |
| 12^{th} month | Largest single | 0.14 | 0.24 |
| | Impurity | | |
| | Total impurities | 0.31 | 0.51 |

According to the stability investigation results of long-term and accelerated tests, Embodiment 7 has higher stability compared with Comparative example 3. The related substances of the product may be affected by temperature and humidity, so the product should be stored in a cool place.

The above is merely illustrative of the preferred embodiments of the present application and is not intended to limit the present application, and various changes and modifications may be made to the present application by those skilled in the art. Any modifications, equivalent substitutions, improvements and the like made within the spirit and scope of the present application should be included within the protection scope of the present application.

1-10. (canceled)

11. A pharmaceutical composition, wherein the pharmaceutical composition is a granule, and the granule comprises:
an active ingredient, comprising 2-(2-aminopyridine-4-yl)-5-(5-((5-(2-ethyl-2H-tetrazole-5-yl)pyridine-2-yl)-oxy)-3,3-amyldimethyl)-1,2,5-thiadiazolidin 1,1-dioxide or pharmaceutically acceptable salt thereof; and
auxiliary materials, comprising a diluent and a suspending agent, the suspending agent comprising povidone.

12. The pharmaceutical composition according to claim 10, wherein the suspending agent further comprises one or more of carboxymethylcellulose sodium, carrageenan or crospovidone (CL-M).

13. The pharmaceutical composition according to claim 10, wherein the pharmaceutically acceptable salt comprises hydrochloride, sulfate, citrate, benzenesulfonate, hydrobromide, hydrofluoric acid, phosphate, acetate, propionate, succinate, oxalate, malate, fumarate, maleate, tartrate or trifluoroacetate.

14. The pharmaceutical composition according to claim 10, wherein the povidone is povidone K30 or povidone K90.

15. The pharmaceutical composition according to claim 10, further comprising a solubilizer, wherein the solubilizer is preferably poloxamer 188 and/or polysorbate 80; and the solubilizer preferably accounts for 0.5% to 2.0% of a total weight of the pharmaceutical composition.

16. The pharmaceutical composition according to claim 10, wherein the diluent comprises one or more of sucrose, mannitol, sorbitol and xylitol.

17. The pharmaceutical composition according to claim 10, wherein the auxiliary materials further comprise essence; the essence comprises one or more of orange essence, strawberry essence and banana essence; and the essence preferably accounts for 0% to 1.0% of a total weight of the pharmaceutical composition.

18. The pharmaceutical composition according to claim 10, wherein the active ingredient accounts for 1.0% to 3.0% of a total weight of the pharmaceutical composition; the diluent accounts for 60% to 98% of the total weight of the pharmaceutical composition; and the suspending agent accounts for 0.3% to 2.0% of the total weight of the pharmaceutical composition.

19. A method for preparing the pharmaceutical composition according to claim 10, comprising:
a) obtaining an adhesive;
b) mixing an active ingredient and a filling agent, and adding the adhesive for granulation;
c) performing dynamic drying; and
d) performing granule arrangement and total mixing.

20. The method according to claim 19, wherein when the pharmaceutical composition is selected from a granule, the method comprises:
a) adding 0.4 parts of poloxamer 188 and 0.3 parts of povidone K90 to an 80% ethanol solution for uniform mixing to serve as the adhesive;
b) weighing and uniformly mixing 2-(2-aminopyridine-4-yl)-5-(5-((5-(2-ethyl-2H-tetrazole-5-yl)pyridine-2-yl)-oxy)-3,3-amyldimethyl)-1,2,5-thiadiazolidin 1,1-dioxide hydrochloride, mannitol, sorbitol and crospovidone (CL-M) according to a weight ratio of 1:26:12:0.4 by part, and then adding the adhesive to obtain a soft material, and performing sieving with a 20-mesh sieve for granulation;
c) performing drying at 50° C.; and
d) performing granule arrangement with a 20-mesh sieve, adding essence to the granules after granule arrangement for total mixing, and packaging.
